(19) United States
(12) Patent Application Publication
(10) Pub. No.: US 2025/0340579 A1
(43) Pub. Date: Nov. 6, 2025

(54) METHODS FOR COBINAMIDE SYNTHESIS

(71) Applicant: The Regents of the University of California, Oakland, CA (US)

(72) Inventors: Gerard Boss, La Jolla, CA (US); Adriano Chan, San Diego, CA (US)

(21) Appl. No.: 18/868,132

(22) PCT Filed: May 26, 2023
(86) PCT No.: PCT/US2023/023656

§ 371 Date: Nov. 21, 2024

Related U.S. Application Data
(60) Provisional application No. 63/346,599, filed on May 27, 2022.

(51) Int. Cl. C07F 15/06

(52) U.S. Cl. CPC C07F 15/065

(57) ABSTRACT

Methods of synthesis of cobinamide compounds with improved yield and purity. Methods and compositions for treating cyanide, sulfide, sodium azide, or methane-thiol exposure in a subject.

### CROSS-REFERENCE TO RELATED APPLICATIONS

This application claims priority to U.S. Provisional Patent Application No. 63/346,599, filed May 27, 2022, which is incorporated by reference herein.

### BACKGROUND

Cyanide is a highly toxic agent that inhibits mitochondrial cytochrome-c oxidase, thereby depleting cellular ATP. Cyanide exposure typically contributes to smoke inhalation deaths in fires, and could be used as a weapon of mass destruction. Cobalamin (vitamin B₁₂) binds cyanide with a relatively high affinity, and is used in Europe to treat smoke inhalation victims. Cobalamin also is FDA-approved for cyanide treatment in the USA. Cobinamide, the penultimate compound in cobalamin biosynthesis, binds cyanide with about 10¹⁰ greater affinity than cobalamin, and can be as effective when administered up to 5 minutes post-cyanide exposure as when given pre-exposure. Cobinamide also can be an effective intra- and/or extra-cellular nitric oxide scavenger.

Three cyanide antidotes are currently available: nitrites, thiosulfate, and hydroxocobalamin. All three drugs are approved only for intravenous (IV) administration, and thus are not suitable for treating mass casualties that could occur in certain circumstances, such as after a major industrial accident or a terrorist attack. In addition to inhalational exposure, people could be exposed to cyanide by eating or drinking cyanide-contaminated food or water. The currently available drugs for treating cyanide exposure, such as hydroxocobalamin (CYANOKIT® hydroxocobalamin for injection, MERIDIAN MEDICAL TECHNOLOGIES®, USA) and sodium thiosulfate/sodium nitrite (NITHIODOTE®, HOPE PHARMACEUTICALS®, USA), have to be administered intravenously, thereby requiring competent medical personnel to administer the drugs properly to a subject immediately at the time and place of cyanide exposure. Additionally, intravenous administration is a time consuming method of administering an antidote.

Therefore, there remains a need for improved methods to synthesize cobinamide compounds, such as aquohydroxo-cobinamide.

### SUMMARY OF THE INVENTION

Provided herein are methods of synthesis of cobinamide compounds and pharmaceutical compositions containing the same. The disclosure provides a synthesis of aquohydroxo-cobinamide with improved yield and purity. Yield and purity may be determined, for example, by high performance liquid chromatography and mass spectrometry.

The invention provides methods of synthesizing aquohydroxo-cobinamide from hydroxo-cobalamin. In embodiments, the invention produces high purity aquohydroxo-cobinamide from hydroxo-cobalamin as described herein. The invention further provides using cerium hydroxide to hydrolyze the phosphoester bond of hydroxo-cobalamin, and then purifying the resulting cobinamide product over two, or more, successive reversed phase resin columns.

The method results in a higher yield and greater purity of the aquohydroxo-cobinamide product than previously described methods. Previously described are methods to produce dicyano-cobinamide (Methods Enzymology 18c: 82-86, 1971). Efficiently converting dicyano-cobinamide to aquohydroxo-cobinamide has previously been unsuccessful. The present method of synthesis provides a yield of at least about 20%, 25%, 30%, 35% or 40%, or greater. The present method of synthesis provides a product purity of at least about 80% 85%, 90%, 95%, 96%, 97%, 98%, or greater, as assessed by high performance liquid chromatography and mass spectrometry.

The compounds and/or compositions can be used in methods for treating cyanide, sulfide, sodium azide, and/or methane thiol exposure in a subject. In one aspect, methods for treating cyanide, sulfide, sodium azide, and methane thiol exposure in a subject are provided. A subject is “exposed” to one of the foregoing, e.g., sodium azide, when the subject contacts, in any manner (e.g., inhales), one or more of the foregoing. e.g., sodium azide, or a product thereof (e.g., hydrazoic acid, which may be produced when sodium azide contacts water). The compounds and/or compositions also may be administered as an antioxidant (see, e.g., Schwarzer, G. et al. Nature Communications, 2019, 10, 3533; and Chang, S. et al. PNAS Nexus, 2022, 1 (4), 191). In some embodiments, the methods include administering a therapeutically effective amount of a cobinamide compound to a subject. A cobinamide compound may be a component of a pharmaceutical composition.

In some embodiments, the cobinamide compound is administered at a dose of about 1 mg/kg to about 600 mg/kg, about 1 mg/kg to about 500 mg/kg, about 1 mg/kg to about 400 mg/kg, about 1 mg/kg to about 300 mg/kg, about 1 mg/kg to about 200 mg/kg, about 1 mg/kg to about 100 mg/kg, about 1 mg/kg to about 75 mg/kg, about 1 mg/kg to about 50) mg/kg, about 1 mg/kg to about 24 mg/kg, or about 1 mg/kg to about 16 mg/kg.

In some embodiments, the pharmaceutical compositions provided herein are administered, or are formulated to be administered, by intramuscular injection to neutralize cyanide, sodium azide, sulfide, and/or methane thiol. The pharmaceutical compositions may be administered by intramuscular injection in order to rescue a subject from lethal poisoning by these toxic chemicals.

In some embodiments, the pharmaceutical compositions provided herein include one or more cobinamide compounds, and are formulated for delivery to a subject, wherein the pharmaceutical compositions are effective for treating a disease state in a subject caused or exacerbated by the presence of excess nitric oxide (NO) in the subject.

In some embodiments, the pharmaceutical compositions provided herein include one or more cobinamide compounds, and are formulated for delivery (e.g., intramuscular delivery) to a subject, wherein the pharmaceutical compositions are effective to neutralize cyanide, sodium azide, sulfide, and/or methane thiol in the subject.

In some embodiments, the one or more cobinamide compounds include an amino-tetrazole-cobinamide, a di-(amino-tetrazole)-cobinamide, an acetyl-tetrazole-cobinamide, a di-(acetyl-tetrazole)-cobinamide, an acetyl-imidazole-cobinamide, a di-(acetyl-imidazole)-cobinamide, or a combination thereof. An example of an amino-tetrazole-cobinamide is 5-amino-tetrazole-cobinamide. An example of a di-(amino-tetrazole)-cobinamide is di-(5-amino-tetrazole)-cobinamide. An example of an acetyl-tetrazole-cobinamide is 5-acetyl-tetrazole-cobinamide. An example of a di-(acetyl-tetrazole)-cobinamide is di-(5-acetyl-tetrazole)-cobinamide. An example of an acetyl-imidazole-cobinamide is 4-acetyl-imidazole-cobinamide. An example of a di-(acetyl-imidazole)-cobinamide is di-(4-acetyl-imidazole)-cobinamide.

In some embodiments, the cobinamide compounds are safe and/or easy to administer by intramuscular injection to one or more subjects at the site of cyanide, hydrogen sulfide, sodium azide, or methane thiol poisoning. Compared to other forms of cobinamide that have been developed, the cobinamide compounds presented herein, in some embodiments, are much safer in subjects.

### BRIEF DESCRIPTION OF THE DRAWINGS

FIG. 1 depicts structures of hydroxocobalamin (top left), aquohydroxocobinamide (top right), and embodiments of ligands (bottom).

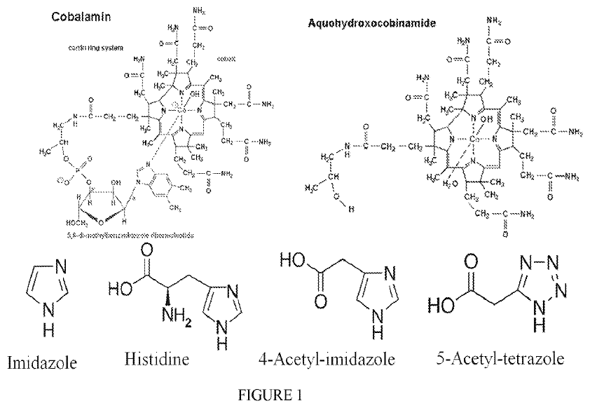

### DETAILED DESCRIPTION

All publications, patents, and patent applications mentioned in this specification are hereby incorporated by reference to the same extent as if each individual publication, patent, or patent application was specifically and individually indicated to be incorporated by reference.

The practice of the present invention will employ, unless otherwise indicated, conventional techniques of molecular biology (including recombinant techniques), microbiology, cell biology, biochemistry and immunology, which are known to and employable by those of ordinary skill in the art.

### Definitions

To facilitate understanding of the invention, a number of terms and abbreviations as used herein are defined below as follows:

When introducing elements of the present invention or the embodiment(s) thereof, the articles “a” “an.” “the,” and “said” are intended to mean that there are one or more of the elements. The terms “comprising,” “including,” and “having” are intended to be inclusive and mean that there may be additional elements other than the listed elements.

The term “and/or” when used in a list of two or more items, means that any one of the listed items can be employed by itself or in combination with any one or more of the listed items. For example, the expression “A and/or B” is intended to mean either or both of A and B, i.e. A alone, B alone, or A and B in combination. The expression “A, B, and/or C” is intended to mean A alone, B alone, C alone, A and B in combination, A and C in combination, B and C in combination, or A, B, and C in combination.

It is understood that aspects and embodiments of the invention described herein include “consisting” and/or “consisting essentially of” aspects and embodiments.

It should be understood that the description in range format is merely for convenience and brevity and should not be construed as an inflexible limitation on the scope of the invention. Accordingly, the description of a range should be considered to have specifically disclosed all the possible sub-ranges as well as individual numerical values within that range. For example, description of a range such as from about 1 to about 6 should be considered to have specifically disclosed sub-ranges such as from about 1 to about 3, from about 1 to about 4, from about 1 to about 5, from about 2 to about 4, from about 2 to about 6, from about 3 to about 6 etc., as well as individual numbers within that range, for example, about 1, about 2, about 3, about 4, about 5, and about 6. This applies regardless of the breadth of the range. Values or ranges may be also be expressed herein as “about,” from “about” one particular value, and/or to “about” another particular value. When such values or ranges are expressed, other embodiments disclosed include the specific value recited, from the one particular value, and/or to the other particular value. Similarly, when values are expressed as approximations, by use of the antecedent “about,” it will be understood that the particular value forms another embodiment. It will be further understood that there are a number of values disclosed therein, and that each value is also herein disclosed as “about” that particular value in addition to the value itself. In embodiments, “about” can be used to mean, for example, within 10% of the recited value, within 5% of the recited value, or within 2% of the recited value.

As used herein, “patient” or “subject” means a human or animal subject to be treated.

As used herein the term “pharmaceutical composition” refers to pharmaceutically acceptable compositions, wherein the compositions include one or more cobinamide compounds, and, in some embodiments, also includes a pharmaceutically acceptable carrier. In some embodiments, the pharmaceutical composition is a combination.

As used herein the term “pharmaceutically acceptable” means approved by a regulatory agency of the Federal or a state government or listed in the U.S. Pharmacopoeia, other generally recognized pharmacopoeia in addition to other formulations that are safe for use in animals, and more particularly in humans and/or non-human mammals.

As used herein the term “pharmaceutically acceptable carrier” refers to an excipient, diluent, preservative, solubilizer, emulsifier, adjuvant, and/or vehicle with which one or more cobinamide compounds may be administered. Such carriers may be sterile liquids, such as water and oils, including those of petroleum, animal, vegetable, or synthetic origin, such as peanut oil, soybean oil, mineral oil, sesame oil and the like, polyethylene glycols, glycerine, propylene glycol or other synthetic solvents. Antibacterial agents such as benzyl alcohol or methyl parabens: antioxidants such as ascorbic acid or sodium bisulfite: chelating agents such as ethylenediaminetetraacetic acid; and agents for the adjustment of tonicity such as sodium chloride or dextrose may also be a carrier. Methods for producing compositions in combination with carriers are known to those of skill in the art. In some embodiments, the language “pharmaceutically acceptable carrier” is intended to include any and all solvents, dispersion media, coatings, isotonic and absorption delaying agents, and the like, compatible with pharmaceutical administration. The use of such media and agents for pharmaceutically active substances is well known in the art. See, e.g., Remington, The Science and Practice of Pharmacy, 20th ed., (Lippincott, Williams & Wilkins 2003). Except insofar as any conventional media or agent is incompatible with the active compound, such use in the compositions is contemplated.

As used herein, the phrases “effective amount,” “therapeutically effective amount,” or the like refer to an amount of one or more cobinamide compounds that is sufficient to treat or ameliorate, or in some manner reduce the symptoms associated with cyanide, sulfide, sodium azide, and/or methane thiol exposure. When used with reference to a method, the method is sufficiently effective to treat or ameliorate, or in some manner reduce the symptoms associated with cyanide, sulfide, sodium azide, and/or methane thiol exposure. For example, an effective amount in reference to cyanide, sulfide, sodium azide, and/or methane thiol exposure is that amount which is sufficient to neutralize, block, or prevent onset of the adverse effects of cyanide, sulfide, sodium azide, and/or methane thiol exposure: or if symptoms have begun, to palliate, ameliorate, stabilize, reverse or slow progression of the adverse effects, or otherwise reduce pathological consequences of cyanide, sulfide, sodium azide, and/or methane thiol exposure. In any case, an effective amount may be given in single or divided doses.

As used herein, the terms “treatment,” “treating,” or the like embrace at least an amelioration of the symptoms associated with cyanide, sulfide, sodium azide, and/or methane thiol exposure in the patient, where amelioration is used in a broad sense to refer to at least a reduction in the magnitude of a parameter, e.g. a symptom associated with cyanide, sulfide, sodium azide, and/or methane thiol exposure being treated. As such, “treatment,” “treating,” or the like also include situations where cyanide, sulfide, sodium azide, and/or methane thiol exposure, or at least symptoms associated therewith, are completely inhibited (e.g. prevented from happening) or stopped (e.g. terminated) such that the patient no longer suffers from the adverse effects associated with cyanide, sulfide, sodium azide, and/or methane thiol exposure, or at least the symptoms that characterize cyanide, sulfide, sodium azide, and/or methane thiol exposure.

The term “combination” refers to either a fixed combination in one dosage unit form, or a kit of parts for the combined administration where one or more cobinamide compounds and a combination partner (e.g., another drug as explained below, also referred to as “therapeutic agent” or “co-agent”) may be administered independently at the same time or separately within time intervals. In some circumstances, the combination partners show a cooperative, e.g., synergistic effect. The terms “co-administration” or “combined administration” or the like as utilized herein are meant to encompass administration of the selected combination partner to a single subject in need thereof, and are intended to include treatment regimens in which the agents are not necessarily administered by the same route of administration or at the same time. The term “pharmaceutical combination” as used herein means a product that results from the mixing or combining of more than one active ingredient and includes both fixed and non-fixed combinations of the active ingredients. The term “fixed combination” means that the active ingredients, e.g., a compound and a combination partner, are both administered to a patient simultaneously in the form of a single entity or dosage. The term “non-fixed combination” means that the active ingredients, e.g., a compound and a combination partner, are both administered to a patient as separate entities either simultaneously, concurrently or sequentially with no specific time limits, wherein such administration provides therapeutically effective levels of the two compounds in the body of the patient. The latter also applies to cocktail therapy, e.g., the administration of three or more active ingredients.

As used herein, the phrase “cobinamide compound” refers to cobinamide and/or cobinamide derivatives. A cobalt atom of the cobinamide and cobinamide derivatives independently may be coordinated with one or more ligands, typically two ligands. As used herein, the phrase “cobinamide derivative” refers to a biologically active derivative (e.g., analog) of cobinamide, such as a heterocyclic or heteropolycyclic compound that is (i) coordinated with a central cobalt atom, and (ii) substituted with two or more alkyl substituents (e.g., four to eight alkyl substituents) that include at least one polar functional group, such as an amide, an ester, an ether, carboxylic acid, etc. The heterocyclic or heteropolycyclic compound may include 4 heteroatoms, such as nitrogen, oxygen, etc. The ligands of the cobinamide compounds disclosed herein may include any ligand that is capable of coordinating with the cobalt atom, such as an unsubstituted or substituted tetrazole, an unsubstituted or substituted imidazole, histidine, etc. As an example, a cobinamide compound may include 5-amino-tetrazole-hydroxo-cobinamide, which is cobinamide coordinated with one 5-amino-tetrazole ligand, or di-(5-amino-tetrazole)-cobinamide, which is cobinamide coordinated with two 5-amino-tetrazole ligands. As used herein, the phrase “amino-tetrazole” refers to a tetrazole moiety substituted at any one or more positions with (i) an amino moiety and/or (ii) a C₁-C₃ alkyl comprising an amino moiety. As used herein, the phrase “acetyl-tetrazole” refers to a tetrazole moiety substituted at any one or more positions with (i) an acetyl moiety and/or (ii) a C₁-C₃ alkyl comprising an acetyl moiety. As used herein, the phrase “acetyl-imidazole” refers to an imidazole moiety that is substituted at any one or more positions with (i) an acetyl moiety and/or (ii) a C₁-C₃ alkyl comprising an acetyl moiety.

Provided herein are pharmaceutical compositions that may include one or more cobinamide compounds, and methods of using the pharmaceutical compositions as a cyanide, sulfide, or methane-thiol antidote. In some embodiments, the one or more cobinamide compounds include two ligands, which may improve the safety profile of a cobinamide complex and/or allow the one or more cobinamide compounds to be absorbed (completely or partially) after intramuscular injection. In some embodiments, the one or more cobinamide compounds have a low pKa for dissociation of one or more ionizable protons. In preferred embodiments, an unsubstituted or substituted tetrazole, which resembles an imidazole but has a pKa of 4.7 instead of 7.0, is the ligand of the one or more cobinamide compounds. When the ligands include amino-tetrazole and/or acetyl-tetrazole, the pKa of the nitrogen that coordinates to the cobalt may be about 4.9, which is about two orders of magnitude lower than for imidazole-containing cobinamides. This lower pKa can markedly increase acetyl-tetrazole and/or amino-tetrazole binding to cobinamide under physiological conditions.

### Embodiments

The following is a non-limiting listing of embodiments of the disclosure:

Embodiment 1. A method of synthesis, the method comprising contacting a hydroxo-cobalamin and a hydrolyzing agent to form an aquohydroxo-cobinamide.

Embodiment 2. The method of Embodiment 1, wherein the aquohydroxo-cobinamide is present in a mixture, and the mixture may also include dimethylbenzimidazole.

Embodiment 3. The method of Embodiment 2, wherein the mixture further comprises the hydrolyzing agent, a portion of the hydroxo-cobalamin that is not hydrolyzed by the contacting of the hydroxo-cobalamin and the hydrolyzing agent, or a combination thereof.

Embodiment 4. The method of any of the preceding embodiments, wherein the hydrolyzing agent hydrolyzes at least one phosphoester bond of the hydroxo-cobalamin.

Embodiment 5. The method of any of the preceding embodiments, wherein the hydrolyzing agent is a metal hydroxide, such as cerium hydroxide.

Embodiment 6. The method of any of the preceding embodiments, wherein the aquohydroxo-cobinamide is produced at a yield of at least about 20%, at least about 25%, at least about 30%, at least about 35%, at least about 40%, at least about 45%, or at least about 50%.

Embodiment 7. The method of any of the preceding embodiments, wherein the aquohydroxo-cobinamide, after the purifying of the aquohydroxo-cobinamide, has a product purity of at least about 80%, at least about 85%, at least about 90%, at least about 95%, at least about 96%, at least about 97%, or at least about 98%, by weight, as assessed by high performance liquid chromatography and/or mass spectrometry.

Embodiment 8. The method of any of the preceding embodiments, wherein the contacting of the hydroxo-cobalamin and the hydrolyzing agent occurs in an aqueous liquid.

Embodiment 9. The method of any of the preceding embodiments, wherein the contacting of the hydroxo-cobalamin and the hydrolyzing agent occurs at a pH of about 6 to about 8, or about 7.

Embodiment 10. The method of any of the preceding embodiments, wherein the contacting of the hydroxo-cobalamin and the hydrolyzing agent occurs, at least partially, at a temperature of at least 50° C., a temperature of about 40° C. to about 100° C., about 50° C. to about 80° C., or about 60° C. to about 70° C.

Embodiment 11. The method of any of the preceding embodiments, wherein the contacting of the hydroxo-cobalamin and the hydrolyzing agent occurs for at least 1 hour, at least 5 hours, at least 10 hours, at least 15 hours, or at least 20 hours.

Embodiment 12. The method of any of the preceding embodiments, further comprising purifying the aquohydroxo-cobinamide.

Embodiment 13. The method of any of the preceding embodiments, wherein the purifying of the aquohydroxo-cobinamide comprises separating the aquohydroxo-cobinamide from the mixture to form a purified aquohydroxo-cobinamide, such as by subjecting the mixture comprising the aquohydroxo-cobinamide to chromatography.

Embodiment 14. The method of any of the preceding embodiments, wherein the purifying of the aquohydroxo-cobinamide comprises centrifuging the mixture to produce a supernatant, the supernatant comprising a reduced amount (e.g., none, less than 10 wt %, less than 5 wt %, or less than 1 wt %) of the hydrolyzing agent.

Embodiment 15. The method of Embodiment 14, further comprising centrifuging the supernatant to produce a second supernatant, and optionally adjusting the supernatant's pH to about 7 to about 8 prior to the centrifuging.

Embodiment 16. The method of Embodiment 14 or 15, further comprising aerating (e.g., stirring) the supernatant or the second supernatant.

Embodiment 17. The method of Embodiment 16, wherein the aerating is effective to re-oxidize a cobinamide compound to Cbi⁺³.

Embodiment 18. The method of any one of Embodiments 15 to 17, further comprising centrifuging the second supernatant to produce a third supernatant: optionally concentrating the third supernatant: optionally contacting the third supernatant with a base to increase its pH to about 8 to about 10; and applying the third supernatant to at least one chromatography column.

Embodiment 19. The method of any of the preceding embodiments, wherein the at least one chromatography column comprises two or more successive reversed phase resin columns.

Embodiment 20. The method of any of the preceding embodiments, wherein the purifying of the aquohydroxo-cobinamide comprises reducing a pH of an aqueous liquid in which the aquohydroxo-cobinamide is disposed to about 1 to about 4 (or another pH effective to remove bound ammonia from the aquohydroxo-cobinamide), optionally prior to being applied to one of the at least one chromatography columns.

Embodiment 21. The method of any of the preceding embodiments, further comprising lyophilizing the aquohydroxo-cobinamide or the purified aquohydroxo-cobinamide. Embodiment 22. A method of forming a cobinamide compound, the method comprising providing a first aqueous liquid comprising an aquohydroxo-cobinamide, such as the aquohydroxo-cobinamide prepared by the method of any of the preceding embodiments; providing a second aqueous liquid comprising 5-aminotetrazolate-deoxyribose; and contacting the first aqueous liquid and the second aqueous liquid to form a mixture comprising the cobinamide compound.

Embodiment 23. The method of Embodiment 22, wherein the aquohydroxo-cobinamide is present in the first aqueous liquid at a concentration about 350 mM to about 450 mM, or about 400 mM.

Embodiment 24. A cobinamide compound made according to a method of any of the preceding embodiments.

Embodiment 25. A pharmaceutical composition comprising a cobinamide compound, such as a cobinamide compound formed by any method of the preceding embodiments.

Embodiment 26. The pharmaceutical composition of any of the preceding embodiments, further comprising a pharmaceutically acceptable carrier.

Embodiment 27. The pharmaceutical composition of Embodiment 26, wherein the pharmaceutically acceptable carrier comprises an excipient, a diluent, a preservative, or a combination thereof.

Embodiment 28. The pharmaceutical composition of any of the preceding embodiments, wherein the pharmaceutical composition is formulated for intramuscular injection.

Embodiment 29. A method of treatment, the method comprising administering to a patient an effective amount of a cobinamide compound (e.g., a cobinamide compound formed by a method of any of the preceding embodiments) or a pharmaceutical composition of any of the preceding embodiments.

Embodiment 30. The method of any of the preceding embodiments, wherein the patient has been exposed to cyanide, sulfide, sodium azide, methane thiol, nitric oxide, or a combination thereof.

Embodiment 31. The method of any of the preceding embodiments, wherein the cobinamide compound is administered at a dose of about 1 mg/kg to about 600 mg/kg, about 1 mg/kg to about 500 mg/kg, about 1 mg/kg to about 400 mg/kg, about 1 mg/kg to about 300 mg/kg, about 1 mg/kg to about 200 mg/kg, about 1 mg/kg to about 100 mg/kg, about 1 mg/kg to about 75 mg/kg, about 1 mg/kg to about 50 mg/kg, about 1 mg/kg to about 24 mg/kg, or about 1 mg/kg to about 16 mg/kg.

Embodiment 32. The method of any of the preceding embodiments, wherein the cobinamide compound or the pharmaceutical composition is administered intramuscularly.

Embodiment 33. The method, the cobinamide compound, and/or the pharmaceutical composition of any of the preceding embodiments, wherein the cobinamide compound comprises an amino-tetrazole-cobinamide, a di-(amino-tetrazole)-cobinamide, an acetyl-tetrazole-cobinamide, a di-(acetyl-tetrazole)-cobinamide, an acetyl-imidazole-cobinamide, a di-(acetyl-imidazole)-cobinamide, or a combination thereof.

Embodiment 34. The method, the cobinamide compound, and/or the pharmaceutical composition of any of the preceding embodiments, wherein the amino-tetrazole-cobinamide is 5-amino-tetrazole-cobinamide.

Embodiment 35. The method, the cobinamide compound, and/or the pharmaceutical composition of any of the preceding embodiments, wherein the di-(amino-tetrazole)-cobinamide is di-(5-amino-tetrazole)-cobinamide.

Embodiment 36. The method, the cobinamide compound, and/or the pharmaceutical composition of any of the preceding embodiments, wherein the acetyl-tetrazole-cobinamide is 5-acetyl-tetrazole-cobinamide.

Embodiment 37. The method, the cobinamide compound, and/or the pharmaceutical composition of any of the preceding embodiments, wherein the di-(acetyl-tetrazole)-cobinamide is di-(5-acetyl-tetrazole)-cobinamide.

Embodiment 38. The method, the cobinamide compound, and/or the pharmaceutical composition of any of the preceding embodiments, wherein the acetyl-imidazole-cobinamide is 4-acetyl-imidazole-cobinamide.

Embodiment 39. The method, the cobinamide compound, and/or the pharmaceutical composition of any of the preceding embodiments, wherein the di-(acetyl-imidazole)-cobinamide is di-(4-acetyl-imidazole)-cobinamide.

### EXAMPLES

The present invention is further illustrated by the following examples, which are not to be construed in any way as imposing limitations upon the scope thereof. On the contrary, it is to be clearly understood that resort may be had to various other aspects, embodiments, modifications, and equivalents thereof which, after reading the description herein, may suggest themselves to one of ordinary skill in the art without departing from the spirit of the present invention or the scope of the appended claims. Thus, other aspects of this invention will be apparent to those skilled in the art from consideration of the specification and practice of the invention disclosed herein.

Synthesis of Aquohydroxo-Cobinamide from Hydroxocobalamin

Hydroxo-cobalamin (OH-Cbl) is hydrolyzed to aquohydroxo-cobinamide (OH—H₂O-Cbi) and dimethylbenzimidazole (DBZ) at neutral pH using cerium hydroxide [Ce(OH)₃]:

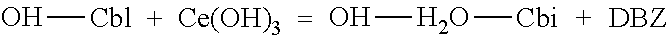

The aquohydroxo-cobinamide is purified from the DBZ and nonhydrolyzed hydroxo-cobalamin on a reverse phase resin (Amberchrom CG-300 M), and then desalted on a second Amberchrom Resin. The purified aquohydroxo-cobinamide is lyophilized to a solid. Start with 20 g hydroxocobalamin: cobinamide yield is ˜6 gram or ˜40%.

### Day 1: Preparation of Cerium Hydroxide and Start of Overnight Hydrolysis of Hydroxocobalamina

Dissolve 225 g CeCl₃ in 250 ml of water in a 1 L flask and degas with nitrogen for at least 20 min: the degassing is necessary to prevent the Ce⁺³ from being oxidized to Ce⁺⁴ during the hydrolysis
Dissolve 93 g NaOH in a beaker with 450 ml of water while stirring
Slowly, with constant stirring, add the NaOH to the CeCl₃ over ˜45 min while degassing to generate Ce(OH)₃; the latter is essentially insoluble in water resulting in a suspension
Stir suspension for an additional hour with degassing to assure complete conversion to Ce(OH)₃
Wash Ce(OH)₃ by centrifugation (10 min×1,500 rpm, IEC centrifuge) with resuspension in degassed water in large tubes: this is to remove excess NaOH
Repeat washing step for ˜4 hours or ˜20 washes to yield a final pH of ˜7.5
Dissolve 20 g of hydroxocobalamin in 75 ml of water in a flask and degas
Resuspend the washed Ce(OH)₃ in 500 ml degassed water and transfer to a screw-capped 1 L flask, degas and heat to 65° C.
Slowly add the hydroxocobalamin to the Ce(OH)₃ suspension while stirring and degassing
Cap the flask and keep at 65° C. while continuously stirring (speed 2-3) overnight for a total of 20 hours
Prepare Amberchrom CG-300 M resin column in 70% ethanol (column dimensions ˜2.6×52 cm)
   Day 2: Recovery of Cobinamide Product from Cerium Hydroxide and Oxidation of Cobinamide
Wash Amberchrom column with water to remove the ethanol, followed by equilibrating it in 10 mM NH₄HCO₃
Centrifuge reaction solution to remove Ce(OH)₃ (10 min×1,500 rpm, IEC)
Collect supernatant and if necessary, adjust pH to 7-8 with NaOH in 500 ml beaker (the pH of the solution falls during the hydrolysis procedure)
Centrifuge at higher speed to remove additional Ce(OH)₃: RC5C with a Sorvall SS-34 rotor (15 min×15,000 rpm, 20° C.)
Transfer supernatant to a large beaker and aerate overnight at room temperature with stirring: most of the cobinamide is reduced from Cbi⁺³ to Chi⁺² during the hydrolysis, and the aeration re-oxidizes it back to Chi⁺³
   Day 3: Conversion of Aquohydroxo-Cobinamide to Di-Amine-Cobinamide and Separation of Di-Amine-Cobinamide from Other Reaction Products on 1^{st} Amberchrom Resin Column
Continue equilibrating Amberchrom column in 10 mM NH₄HCO;
Spin cobinamide preparation once more in RC5C centrifuge with a Sorvall SS-34 rotor (15 min×15,000 rpm, 20° C.): some more Ce(OH); precipitates out during the aeration procedure due to concentration of the solution
Concentrate the di-amine-cobinamide solution to ˜100 ml with rotary evaporator
Add 4.8 g NH₄HCO₃ and adjust pH to 9.0 using NH₄OH: this generates di-amine-cobinamide, which separates better on the Amberchrom resin from the DBZ product than aquohydroxo-cobinamide
Apply the concentrated di-amine-cobinamide solution to Amberchrom column
Elute column with 10 mM NH₄HCO₃, collecting di-amine-cobinamide fraction in ˜500 ml
Store the di-amine-cobinamide solution at 4° C. overnight
Clean Amberchrom column with 70% ethanol (will take multiple days) for reuse
Wash 2^{nd} Amberchrom column (5.3×28 cm) with water to remove ethanol

Day 4: Desalt Purified Aquohydroxo-cobinamide on 2^{nd} Amberchrom Resin
Equilibrate 2^{nd} Amberchrom column in 10 mM HCl (takes ˜1 liter)
Adjust the cobinamide solution to pH 2 using concentrated HCl; this removes bound ammonia to generate ammonium and aquohydroxo-cobinamide
Apply sample to column, and wash column with 1 liter 10 mM HCl and 3 liters of water to remove ammonium and other impurities
Elute purified aquohydroxo-cobinamide in 70% ethanol and store overnight at 4° C.
Wash column with 70% ethanol

### Day 5: Concentration and Lyophilization of Aquohydroxo-Cobinamide to Solid Material

Evaporate ethanol from purified and desalted aquohydroxo-cobinamide preparation by rotary evaporation (concentrate to almost completely dryness)
Reconstitute cobinamide in ˜40 ml of water and transfer to lyophilization jar

Shell freeze and lyophilize the aquohydroxo-cobinamide
Collect dried aquohydroxo-cobinamide and store it at 4° C. (yields approximately 6 g)
   Generation of Bis-Aminotetrazole-Cobinamide from Aquohydroxo-Cobinamide
Dissolve desired amount of solid aquohydroxo-cobinamide in water to a final concentration of ˜400 mM.
Determine actual concentration of the aquohydroxo-cobinamide solution spectrophoto-metrically in a small aliquot by adding 100-fold excess KCN to convert it to dicyano-cobinamide (molar extinction coefficient of dicyano-cobinamide is 3.0×10⁴ M⁻¹ cm⁻¹).
If necessary, add more solid aquohydroxo-cobinamide to the solution to achieve concentration close to 400 mM; then repeat step 2.
Prepare a 4 M solution of 5-aminotetrazolate by dissolving 5-amino-1H-tetrazole in an equivalent amount of NaOH; this is done by dissolving the solid in 10 NaOH, and then adjusting the volume with water; the resulting solution should have a pH˜7.0.
Add solid deoxyribose to the 5-aminotetrazolate solution to a ratio of 8:3, 5-aminotetrazolate to deoxyribose
Add the 5-aminotetrazolate-deoxyribose solution to the aquohydroxocobinamide solution to final concentrations of 8 molar equivalents of 5-aminotetrazolate and 3 molar equivalents of deoxyribose to cobinamide
The pH of the solution can rise due to displacement of the OH-group from the aquohydroxo-cobinamide by the aminotetrazolate, and will need to be down-adjusted to ˜pH 7.4 using 6 M HCl. The pH will continue to rise over the next 24 h, so need to check the pH several times and down adjust with the HCl. Based on the volumes of the 5-aminotetrazolate-deoxyribose and HCl that were added, can calculate volume of water needed to bring solution to 200 mM in terms of cobinamide concentration.
The resulting 200 mM bis-aminotetrazole-cobinamide solution is stable at room temperature when protected from bright light.

1. A method of synthesis, the method comprising:
contacting a hydroxo-cobalamin and a hydrolyzing agent to form a mixture, the mixture comprising an aquohydroxo-cobinamide; and
separating the aquohydroxo-cobinamide from the mixture to obtain a purified aquohydroxo-cobinamide;
wherein the hydrolyzing agent comprises a metal hydroxide,
wherein the aquohydroxo-cobinamide is formed at a yield of at least about 20%, and
wherein the purified aquohydroxo-cobinamide has a purity of at least about 80%, by weight.

2. The method of claim 1, wherein the hydrolyzing agent comprises cerium hydroxide.

3. The method of claim 1, wherein the purified aquohydroxo-cobinamide has a purity of at least about 95%, by weight.

4. The method of claim 1, wherein the contacting of the hydroxo-cobalamin and the hydrolyzing agent occurs (i) in an aqueous liquid, (ii) at a pH of about 7, (iii) at a temperature of at least 50° C., (iv) for at least 5 hours, or (v) a combination thereof.

5. The method of claim 1, wherein the separating of the aquohydroxo-cobinamide from the mixture comprises centrifuging the mixture to produce a supernatant, wherein the supernatant includes an amount of the hydrolyzing agent that is less than an amount of the hydrolyzing agent in the mixture.

6. The method of claim 5, further comprising applying the supernatant to two or more successive reversed phase resin columns.

7. The method of claim 1, further comprising lyophilizing the purified aquohydroxo-cobinamide.

8. A method of forming a cobinamide compound, the method comprising:
providing a first aqueous liquid comprising aquohydroxo-cobinamide;
providing a second aqueous liquid comprising 5-aminotetrazolate-deoxyribose; and
contacting the first aqueous liquid and the second aqueous liquid to form a mixture comprising the cobinamide compound;
wherein the aquohydroxo-cobinamide is present in the first aqueous liquid at a concentration about 350 mM to about 450 mM.

9. A cobinamide compound formed according to the method of claim 1.

10. A pharmaceutical composition comprising:
a cobinamide compound;
wherein the pharmaceutical composition is formulated for intramuscular injection.

11. The pharmaceutical composition of claim 10, wherein the cobinamide compound comprises an amino-tetrazole-cobinamide, a di-(amino-tetrazole)-cobinamide, an acetyl-tetrazole-cobinamide, a di-(acetyl-tetrazole)-cobinamide, an acetyl-imidazole-cobinamide, a di-(acetyl-imidazole)-cobinamide, or a combination thereof.

12. A method of treatment, the method comprising:
administering to a patient an effective amount of a pharmaceutical composition of claim 10;
wherein the patient has been exposed to cyanide, sulfide, sodium azide, methane thiol, nitric oxide, or a combination thereof.

13. The method of claim 12, wherein the cobinamide compound is administered at a dose of about 1 mg/kg to about 600 mg/kg.

14. The method of claim 12, wherein the cobinamide compound or the pharmaceutical composition is administered intramuscularly.
